(19) United States
(12) Patent Application Publication
(10) Pub. No.: US 2024/0274246 A1
(43) Pub. Date: Aug. 15, 2024

(54) PREDICTIVE CLINICAL DATA CONSUMABILITY VALUATION

(71) Applicant: Cerner Innovation, Inc., Kansas City, MO (US)

(72) Inventors: Natalee Agassi, Blue Bell, PA (US); William John Ormerod, JR., Fairfax, VT (US)

(73) Assignee: Cerner Innovation, Inc., Kansas City, MO (US)

(21) Appl. No.: 18/322,282

(22) Filed: May 23, 2023

Related U.S. Application Data
(60) Provisional application No. 63/484,716, filed on Feb. 13, 2023.

(51) Int. Cl. G16H 10/60

(52) U.S. Cl. CPC G16H 10/60

(57) ABSTRACT

Systems and methods determine consumability scores of data to be transformed from a source clinical data schema to a target clinical data schema. Determining the consumability score can include calculating values for characteristics of the source data set transformed from the clinical data schema, weighting the individual scores, and aggregating the weighted scores. The consumability score may indicate a predicted suitability of the transformed data for a target use. Further, the system can generate recommendations for using the target data set based on the consumability score. The determination can include predicting whether the transformation produces elements of a target data set are sufficient for the intended purposes of users of the target data set, whether the source data set includes sufficient information that can be mapped to the target clinical data schema, and whether the transformation captures the source data set in sufficient quantity and quality.

### INCORPORATION BY REFERENCE; DISCLAIMER

The following application is hereby incorporated by reference: application no. 63/484,716, filed Feb. 13, 2023. The applicant hereby rescinds any disclaimer of claims scope in the parent application(s) or the prosecution history thereof and advise the USPTO that the claims in the application may be broader than any claim in the parent application(s).

### TECHNICAL FIELD

The present disclosure relates to processing medical records. More specifically, the present disclosure relates to converting electronic health data between standard data formats.

### BACKGROUND

Health care systems encode health records using standardized electronic formats. For example, when a patient completes a medical encounter with a provider, the provider may generate electronic health data storing clinical information and claims data. The clinical information can include information describing diagnostic testing performed, diagnosis of the patient, and treatment provided. The claims data can include billing codes for the encounter with the provider. The electronic health data can be encoded as structured data objects (e.g., data artifacts) in compliance with one of several standard data formats, such as International Classification of Diseases (ICD), Health Level Seven International (HL7), and Clinical Document Architecture (CCDA), which each can include different versions.

Various entities, such as patients, hospitals, insurance companies, researchers, and regulators may need to convert information stored in electronic health data into different formats usable for their respective purposes. For example, a health care provider may import legacy electronic health data into a data warehouse that uses a different data schema than was originally used to encode the electronic health data. However, importing the electronic health data can be difficult or impossible because a lack of prescriptive data standards for clinical data schemas leaves the data open to a wide variability of interpretations. Consequently, converting information from one standard data schema to another can result in lost information and incomplete records. For example, data encoded in electronic health data using the original schema may be different or incompatible when translated into the updated data schema. Moreover, if the translated electronic health data lacks certain foundational information (e.g., patient name), then data objects depending on that foundational information may be incomplete. Consequently, some records in the translated data may be unusable for some purposes without manual intervention to correct and/or clarify the records.

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 illustrates a functional flow block diagram of an example data transformation system in accordance with one or more embodiments.

FIG. 2 illustrates a block diagram of an example data transformation system in accordance with one or more embodiments.

FIG. 3 illustrates a set of operations of an example process for data transformation in accordance with one or more embodiments.

FIG. 4 illustrates an example of mapping for a data transformation in accordance with one or more embodiments.

FIG. 5 shows a block diagram illustrating an example computer system in accordance with one or more embodiments.

### DETAILED DESCRIPTION

In the following description, for the purposes of explanation, numerous specific details are set forth in order to provide a thorough understanding. One or more embodiments may be practiced without these specific details. Features described in one embodiment may be combined with features described in a different embodiment. In some examples, well-known structures and devices are described with reference to a block diagram form in order to avoid unnecessarily obscuring the present invention.
1. GENERAL OVERVIEW
2. DATA TRANSFORMATION ENVIRONMENT
3. DATA TRANSFORMATION ARCHITECTURE
4. DATA TRANSFORMATION AND CONSUMBILITY VALUATION
5. EXAMPLE CONSUMBILITY VALUATION
6. HARDWARE OVERVIEW
7. MISCELLANEOUS; EXTENSIONS

### 1. GENERAL OVERVIEW

One or more embodiments determine consumability scores of data to be transformed from a source clinical data schema to a target clinical data schema. The consumability score may be based on a transformation process, characteristics of a source data set, and characteristics of a target data set. Determining the consumability score can include calculating values for characteristics of the source data set transformed from the clinical data schema, weighting the individual scores, and aggregating the weighted scores. The consumability score may indicate a predicted suitability of the transformed data for a target user and/or a target use. One or more embodiments generate recommendations for using the target data set, generated from the source data set, based on the consumability score. The determination can include predicting whether the transformation produces elements of a target data set are sufficient for the intended purposes of users of the target data set, whether the source data set includes sufficient information that can be mapped to the target clinical data schema, and whether the transformation captures the source data set in sufficient quantity and quality.

Embodiments enable computing systems to efficiently transform large volumes of complex electronic health records from one standard format to another while generating information about the consumability of the target data set. Determining the consumability information can include generating recommendations indicating whether the computer-generated data is reliable in particular contexts based on the intent of the usage (e.g., clinical, research, caregiver, billing, etc.). For example, embodiments use machine learning models trained to classify the consumability of translated data for an intended purpose based on multiple dimensions of data analysis and generate a display of the consumability along with recommendations for using and/or improving the data. By doing so, embodiments address problems of transforming electronic health records using computer systems that, unlike a human, cannot mentally ascertain the consumability or intended purpose of data. Further, using the consumability information and recommendations, embodiments generate an improved computer-user interface enabling users quickly and efficiently recognize the usefulness of the target data set generated by the computing system.

While this General Overview subsection describes various example embodiments, it should be understood that one or more embodiments described in this Specification or recited in the claims may not be included in this subsection.

### 2. DATA TRANSFORMATION ENVIRONMENT

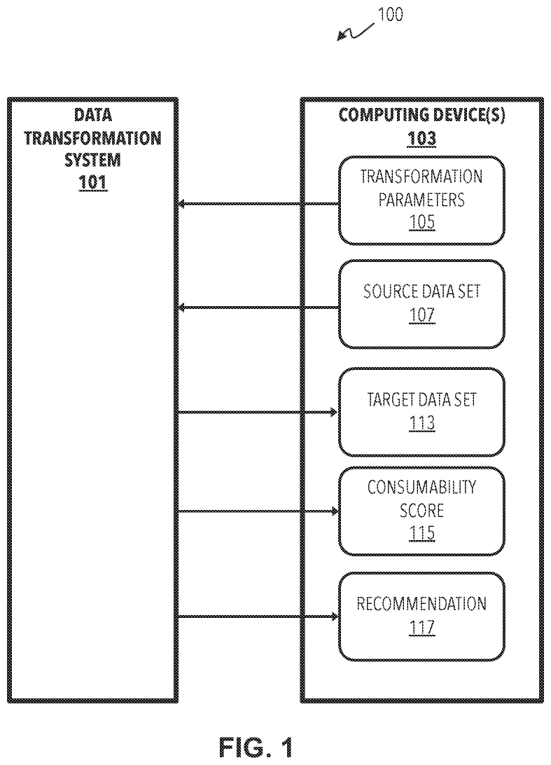

FIG. 1 shows a functional flow block diagram illustrating an example environment 100 for implementing systems and processes in accordance with one or more embodiments. The environment 100 includes a data transformation system 101 and one or more computing devices 103. The computing devices 103 can be communicatively connected, directly or indirectly, to the data transformation system 101 via one or more communication channels, which can be wired or wireless data links and/or a communication networks, such as local area networks, peer-to-peer networks, wide area networks, telephone networks, and the Internet.

The data transformation system 101 can be one or more computing systems that translate a source data set 107 encoded in a standard clinical data schema to a target data set 113 encoded in a different standard clinical data schema based on transformation parameters 105. Additionally, the data transformation system 101 can generate a consumability score 115 for the target data set 113 indicating the suitability of the target data set 113 for an intended use. Further, the data transformation system 101 can generate a recommendation 117 indicating uses and improvements to the target data set 113.

Some embodiments of the computing device 103 can comprise a data repository comprising one or more non-transitory computer-readable, hardware storage devices that store information and computer-readable program instructions used by the processes and functions disclosed herein. For example, the computing device 103 can be a file system, database, collection of tables, or any other storage mechanism storing data. The multiple different storage units and/or devices may or may not be of the same type or located at the same physical site. Further, the computing device 103 may be implemented or executed on the same computing system as the data transformation system 101.

Some embodiments of the computing device 103 allow a user to access and interact with the data transformation system 101 to request transformation of the source data set 107 into a different clinical data schema. The computing device 103 may include a server computer, a personal computer system, a smartphone, a tablet computer, a laptop computer, or other programmable user computing device. The computing device 103 can include one or more processors that process software or other computer-readable program instructions and include a memory to store the software, computer-readable program instructions, and data. The computing device 103 can generate a computer-user interface enabling a user to interact with the computing device 103 and the data transformation system 101 using input/output devices (e.g., keyboard, pointer device, touchscreen, microphone, and speaker). For example, computing device 103 can execute a web browser application that generates an interactive user interface (e.g., a graphic user interface) with which a user can input the transformation parameters 105 interact to request transformation of the source data set 107.

In a non-limiting example, the transformation parameters 105 can identify information for transforming records from a first data schema to a second data schema, including: an identification of the source data set 107, a schema of the source data set 107, a schema of the target data set 113, an intended use (“intent”) of the target data set 113, and type of user of the target data set 113 (e.g., clinician, regulator, billing, and patient). The source data set 107 can be clinical data of one or more patients encoded using the ICD9 standard. The target data set 113 can be the ICD10 standard and the intent can be clinical patient data reconciliation. A user can control the computing device 103 to transmit the transformation parameters 105 and the source data set 107 to the data transformation system 101 for transformation. The user can be, for instance, a data manager migrating patient records from a legacy database of a first healthcare provider to an updated database of a different healthcare provider. Responsive to receiving the source data set 107, the example data transformation system 101 identifies a transformation process for transforming patient data from ICD9 to ICD10. For example, as noted above, the transformation parameters 105 can indicate the clinical data schema of the source data set 107 and/or the target data set 113. Based on the transformation process and the attributes of the data in that first clinical standard, the data transformation system 101 can determine the target data set 113 that would be generated by the transformation based on the target clinical data schema and characteristics of that target data set 113. The system can determine characteristics of the target data set 113 and evaluate the characteristics against the consumability criteria. If the criteria meets or exceeds respective thresholds for an intended use of the target data set, the system can generate and display a consumability score 115 whether to consume the target data set 113. Additionally, the system can generate and display a recommendation 117 indicating how the data should be reviewed and cleaned to improve the consumability score 115.

### 3. DATA TRANSFORMATION ARCHITECTURE

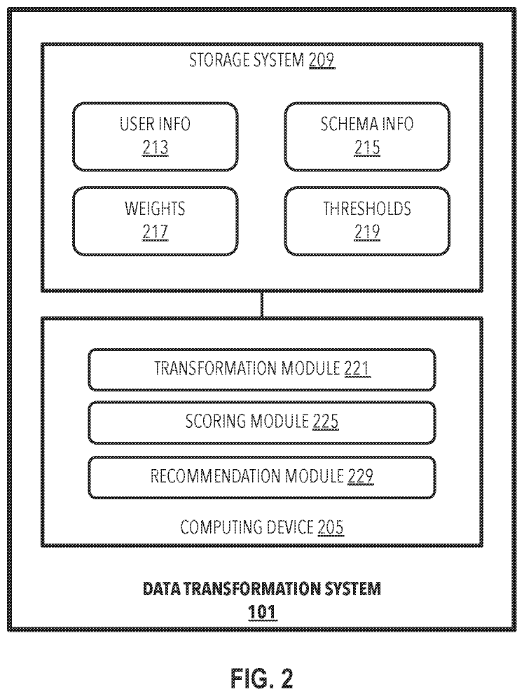

FIG. 2 shows a system block diagram illustrating an example of a data transformation system 101 in accordance with one or more embodiments. The data transformation system 101 can be the same or similar to that described above. The data transformation system 101 includes hardware and software that perform processes and functions disclosed herein. The data transformation system 101 can include a computing device 205 and a storage system 209. The computing device 205 can include one or more processors (e.g., microprocessor, microchip, or application-specific integrated circuit). The storage system 209 can comprise one or more non-transitory computer-readable, hardware storage devices that store information and computer-readable program instructions used by the processes and functions disclosed herein. For example, the storage system 209 can include one or more flash drives and/or hard disk drives.

Additionally, the storage system 209 can store user information 213, schema information 215, weights 217, and thresholds 219 The user information 223 can include profiles describing roles and/or intents of users. For example, the user information 213 can classify a user as an individual data consumer (e.g., a patient), a medical professional, a health care network, a data manager, a data researcher, clinician, an academic, a regulator, or the like. The schema information 215 can be one or more sets of rules or algorithms for mapping and/or deriving elements of standard data schemas from other standard data schema. The weights 217 can be predetermined values for weighting consumability dimensions corresponding to different user roles and/or user intent. The thresholds 219 can be predetermined values for consumability characteristics corresponding to different user roles and/or user intents.

The computing device 205 can execute a transformation module 221, a scoring module 225, and a recommendation module 229, each of which can be software, hardware, or a combination thereof. The transformation module 221 identifies transformation process for transforming data from a source data schema to a target data schema. Additionally, based on the determined transformation process and characteristics of a source data set corresponding to the source data schema, the transformation module 221 determines characteristics of a target data set corresponding to the target clinical standard that can be generated from the source data set. The scoring module 225 determines a consumability score for the target data set by evaluating the characteristics of the target data set based on consumability criteria. The recommendation module 229 determines recommendations for whether a target user should accept the data sets transformed by the transformation module 221. For example, the recommendation module 229 can compare scores to one or more thresholds and, based on determining that a particular threshold is met, recommend consuming the target data set. For example, for a consumability score below a 75 threshold, the recommendation module 229 can recommend manual review of the target data set. Whereas, for a consumability score above an 85% threshold, the recommendation module 229 can recommend automated reconciliation.

It is noted that the data transformation system 101 can comprise any general-purpose computing article of manufacture capable of executing computer program instructions installed thereon (e.g., a personal computer, server, etc.). The data transformation system 101 is representative of various possible equivalent-computing devices that can perform the processes described herein. To this extent, in embodiments, the functionality provided by the data transformation system 101 can be any combination of general and/or specific purpose hardware and/or computer program instructions. In each embodiment, the program instructions and hardware can be created using standard programming and engineering techniques, respectively.

The components illustrated in FIG. 2 may be implemented in software and/or hardware. Each component may be distributed over multiple applications and/or machines. Additionally, it is understood, that one or more of the modules can be stored and executed remotely from the data transformation system 101. Additionally, multiple components may be combined into one application and/or machine. Operations described with respect to one component may instead be performed by another component.

### 4. DATA TRANSFORMATION AND CONSUMABILITY VALUATION

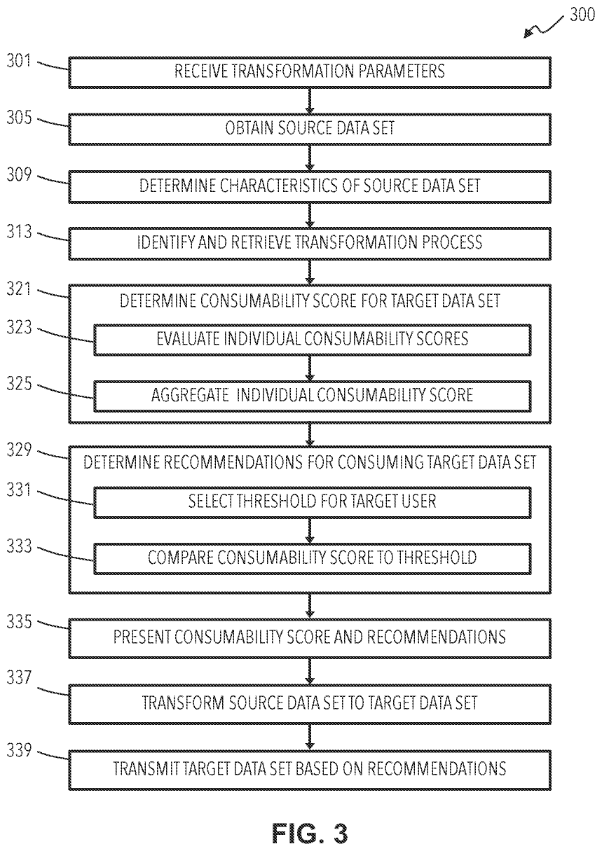

The flow diagram in FIG. 3 illustrates functionality and operations of systems, devices, processes, and computer program products according to various implementations of the present disclosure. Each block FIG. 3 can represent a module, segment, or portion of program instructions, which includes one or more computer executable instructions for implementing the illustrated functions and operations. In some implementations, the functions and/or operations illustrated in a particular block of the flow diagrams can occur out of the order shown in FIG. 3. For example, two blocks shown in succession can be executed substantially concurrently, or the blocks can sometimes be executed in the reverse order, depending upon the functionality involved. Additionally, in some implementations, the blocks of the flow diagrams can be rearranged in different orders. Further, in some implementations, the flow diagram can include fewer blocks or additional blocks. It is also noted that each block of the flow diagrams and combinations of blocks in the flow diagrams can be implemented by special-purpose hardware-based systems that perform the specified functions or acts, or combinations of special-purpose hardware and computer instructions.

FIG. 3 illustrates a set of operations of an example process 300 for transforming data from a source data schema to a target data schema by a computing system (e.g., data transformation system 101) in accordance with one or more embodiments. At block 301, a system (e.g., data transformation system 101) receives transformation parameters (e.g., transformation parameters 105) for the process 300. The transformation parameters can indicate the source data set (e.g., source data set 107), the HL7 v2 data schema of the source data set (“HL7”), the HL7 FHIR schema of the target data set (“FHIR”), a transformation process (e.g., HL7 to FHIR), characteristics of the target data set (e.g., binding requirements), and/or a usage intent (e.g., clinical data, research data, etc.). In some embodiments, the system can receive the transformation parameters through a computer-user interface or a dashboard. For example, the user interface can an interactive graphic user interface including drop-down menus populated with selections for identifying a source data set, a file location of the source data set, a data schema of the source data set, a transformation process, a schema of the target data set, characteristics of the target data set, and the like. In some other embodiments, the system can receive the transformation parameters in a configuration file provided by a user.

At block 305, the system obtains the source data set. For example, the transformation parameters can include information for the system to retrieve the source data set from a data storage system. At block 309, the system (e.g., executing transformation module 221) determines characteristics of the source data set. The characteristics of the source data set can include information obtained at block 301, such as an identification of a data schema used to encode the source data set (e.g., ICD-9), the user type, and the usage intent (e.g., clinical data, research data, etc.). Additionally or alternatively, the system determines the characteristics of the source data set from metadata of the source data set. For example, the source data set can include information identifying the data schema. Additionally, based on the source data schema, the system can infer a user type and/or the usage intent. For example, certain data schema (e.g., SNOWMED) may be primarily for tracking patient encounters and billing. As such, the system can maintain a mapping between certain schema and certain types/intents.

At block 313, the system identifies and retrieves a transformation process for translating the source data set to from a source data schema to a target data schema (e.g., ICD-10). The transformation process can be a set of rules or algorithms (e.g., schema information 215) that map and/or derive elements of the target data schema from the source data schema. Some embodiments identify the source clinical data schema and a target clinical data schema form the parameters received at block 301. Other embodiments can detect the source clinical data schema from the metadata, characteristics, and/or structure of the source data. Based on the identification of the source clinical data schema and the target clinical data schema, the system can retrieve corresponding rules or algorithms for mapping and/or deriving elements for transforming the source data. For example, the user can select the clinical data schema of the source data set and/or the target data set via drop down menus of interactive graphic user interface. Using the information received via the user interface, the system can retrieve a corresponding transformation process.

At block 321, the system (e.g., executing scoring module 225) determines a consumability score for the target data set (e.g., consumability score 115). The consumability score represents a quality of the target data set based on the characteristics of the target data set, including accuracy, integrity, fidelity, and usability. Determining the consumability score can include, at block 323, evaluating individual consumability parameters of the target data set. The consumability parameters can include an accuracy score, an integrity score, a fidelity score, a usability score, and a validity score. The scores can be a value between −1 and 1. In some cases, such as when the evaluation metric is a true/false (e.g., “is a code present?”), the score can be a value between 0 and 1. In other cases, such as binding strength, the score assigned in a range (e.g., required=1, extensible=0.5, sample=0.1, and otherwise=−1). It is understood that other scoring ranges can be used. For example, the consumability parameters can be scored on a range of 1 to 10.

The accuracy score can be a value representing equivalency of the transformation (e.g., matching) between the source data set and the target data set. The accuracy can be scored on whether a structural mapping exists between a code in the source schema and one or more codes in the target schema. For example, if the source schema includes an element encoding result data of a laboratory observation (e.g., “OBX.5—Observation Value”), the system can determine whether the target data schema includes a corresponding element it (e.g., Observation.value_Codeableconcept).

The integrity score can be a value representing completeness of the data. The integrity can be scored based on whether a structural mapping exists between components of the element in the source schema and elements in the corresponding codes in the target schema. For example, the element encoding the result data of the laboratory observation in the source schema includes three components (e.g., code, system, and text), the system can determine whether the corresponding element of the target data schema includes corresponding components (e.g., code, system, and display) such that source information is not lost in the transformation.

The fidelity score can be a value representing whether information in the target data set maintained the same meaning as in the source data set. The fidelity can be scored based on whether the source schema and the target schema use a same encoding dictionary for the code (e.g., SNOMED). The fidelity score can also be based on whether the code of the source schema has a value interpretable by the destination schema. For example, the system can determine that data (e.g., “\hat{ }NEG\hat{ }”) included an element (e.g., “text”) of the code of the source scheme corresponds to a same term or a synonym in the target schema.

The usability score can be a value representing whether data included in the source schema retains functionality in the target schema. For example, the system can determine that data (e.g., “\hat{ }NEG\hat{ }”) included an element (e.g., “text”) of the code is codable concept in the source schema referenced by data processing functions. Whereas, in the target schema, the data is interpreted as plain text lacking any association with data processing functions. The scoring of usability can also be based on a binding strength of the transformed data in the destination schema. Binding can be a rule of the target schema that certain data conform to predefined value sets to different degrees. If binding is required, then the data must conform to the specified value set. If binding is preferred, then the data may draw from the specified codes for interoperability purposes but are not required to do so to be considered conformant. If binding is exemplary, then the data is not required to conform to the specified value set. Instead, the value set merely provides examples of the types of concepts intended to be included.

Determining the consumability score can include, at block 325 aggregating the weighed scores determined at block 323 to determine an aggregated value for the consumability score. Some embodiments determine the consumability score (CS) using the following equation:

$$C S = ( A * W 1 ) + ( I * W 2 ) + ( F * W 3 ) + ( U * W 4 ) \qquad ( 6 )$$

In equation (6) above, W1, W2, W3, and W4 represent respective weights (e.g. 0.2) totaling 1.0 when summed. One or more embodiments select the weights W1, W2, W3, and W4 (e.g., weights 217) having different values based on the intent parameter. The intent parameter can be metadata indicating an intended use of different end users or types of end users (e.g., user information 213). Some embodiments maintain different combinations of weights corresponding to different intent parameters. Based on the intent parameter, the corresponding weights can by applied for determining the consumability score.

Some embodiments also determine a roundtrip score of the target data set indicating. whether the transformed information is translatable back into the source schema without loss of accuracy, fidelity, and usability. The roundtrip score is indicative of the flexibility of the target data set for intents, such as inclusion regulatory reports. The round trip score can be reported along with the aggregated consumability score or the round trip score can be included in the aggregated consumability score.

At block 329, the system determines recommendations for consuming the target information based on the consumability score determined at block 321. Determining the recommendation can include, at block 331, selecting thresholds of the consumability criteria (e.g., thresholds 219) for the target user. Determining the recommendation can also include, at block 333, comparing the consumability score determined at block 321 to the thresholds. For example, responsive to determining that the threshold is met, the system can recommend consuming the target data set. Also, for example, responsive to determining that the threshold is not met, the system can recommend not consuming the target data set and/or recommend clinician review of the target data set. The values of the consumability score can indicate whether: the target data set can be used to make clinical decisions, that the target data set can affect the efficacy of clinical decisions, and the target data set meets an intended functionality.

At block 335, the system presents the consumability score and recommendations. Some embodiments can generate a display using a computer-user interface indicating the consumability score. For example, if the intent is to use for clinical research or a pharmaceutical company research, and based on certain parameters combination the system can determine that the target data set is not usable, because quality if data is insufficient for research; whereas the quality is sufficient for patient consumption. Additionally, the system can exclude certain parameters from the consumability score based on intent. Further, the system can display a ranking of consumability scores for multiple source data sets for same target data set. The display can also include individual consumability parameters of the target data set in comparison to the corresponding thresholds. In some embodiments, if a threshold is not met, the system recommends modifications that result in the greatest reduction or improvement of the consumability score. For example, if the usability elements for accuracy score (A), the integrity score (I), the usability score (U), and the validity score (V) meet or exceed respective thresholds, however), the fidelity score (F) is below a respective threshold, the system. can identify the fidelity factor as causing the greatest reduction of the consumability score. Accordingly, the system could provide a recommendation to improve the fidelity score.

At block 337, the system generates records of the target data schema from one or more records for the source data schema. For example, a record type of the target data schema can include information contained in or derived from multiple records of the source data schema. At block 339, the system transmits the target data set to, for example, the computing device based on the recommendations determined at block 335.

One or more embodiments determine the consumability score using a machine learning model. In one or more embodiments, a machine learning algorithm is an algorithm that can be iterated to learn a target model f that best maps a set of input variables to an output variable. In particular, a machine learning algorithm is configured to generate and/or train a machine learning model. A machine learning algorithm is an algorithm that can be iterated to learn a target model f that best maps a set of input variables (e.g., accuracy, integrity, fidelity, usability, and validity) to an output variable (e.g., aggregated consumability score), using a set of training data (e.g., historical accuracy, integrity, fidelity, usability, and validity). The training data includes datasets and associated labels. The datasets are associated with input variables for the target model f. The associated labels are associated with the output variable of the target model f. The training data may be updated based on, for example, feedback on the accuracy of the current target model f. Updated training data is fed back into the machine learning algorithm, which in turn updates the target model f. A machine learning algorithm generates a target model/such that the target model f best fits the datasets of training data to the labels of the training data. Additionally or alternatively, a machine learning algorithm generates a target model/such that when the target model f is applied to the datasets of the training data, a maximum number of results determined by the target model f matches the labels of the training data. Different target models be generated based on different machine learning algorithms and/or different sets of training data. A machine learning algorithm may include supervised components and/or unsupervised components. Various types of algorithms may be used, such as linear regression, logistic regression, linear discriminant analysis, classification and regression trees, naïve Bayes, k-nearest neighbors, learning vector quantization, support vector machine, bagging and random forest, boosting, backpropagation, and/or clustering. Embodiments train a machine learning model to compute consumability scores using training data sets including characteristics of initial clinical standard characteristics of your final clinical standard characteristics consumability factors. Then the label would be the consumability score.

Additionally, one or more embodiments determine the consumability score (ACS) using clustering techniques. For example the system can generate an N-dimensional cluster where the N dimensions represent the different dimensions and measurements. Using a cartesian distance from a core cluster, the system can determine whether information is withing certain thresholds meeting consumability criteria. For example, to determine a consumability score, the system can determine whether an element is within a certain distance of each other, wherein the core of the cluster would be the Cartesian midpoint of all those within the same cluster.

### 5. EXAMPLE CONSUMABILITY VALUATION

A detailed example is described below for purposes of clarity. Components and/or operations described below should be understood as one specific example which may not be applicable to certain embodiments. Accordingly, components and/or operations described below should not be construed as limiting the scope of any of the claims.

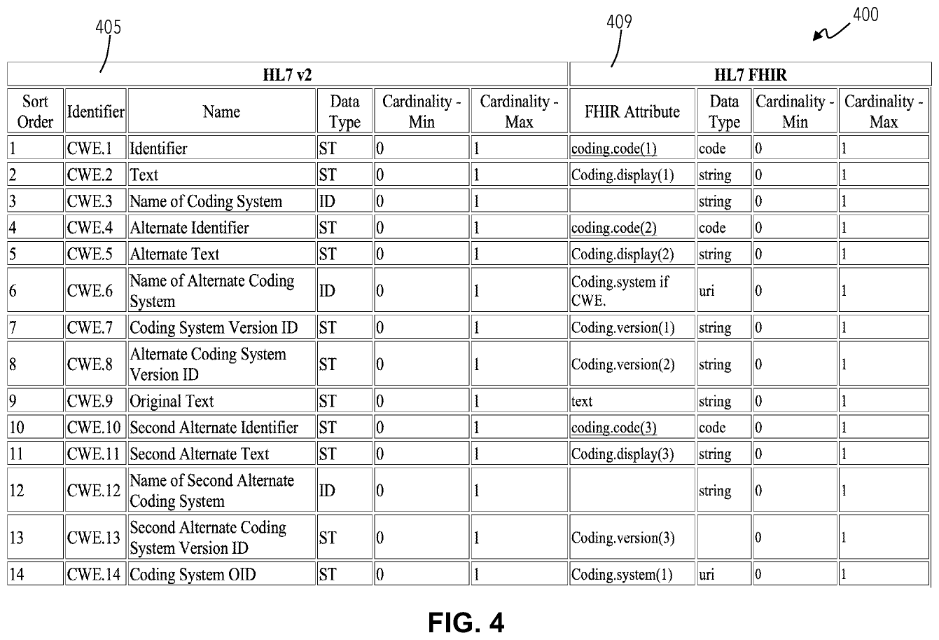

FIG. 4 illustrates an example mapping 400 between a source data schema 405 and a target data schema 409 for a transformation and consumability valuation process (e.g., process 300) in accordance with one or more embodiments. In the present example, the source data schema 405 is encoded using the HL7 v2 standard (“HL7”) and the destination data schema 409 is encoded using the HL7 FHIR standard (“FHIR”). It is understood that the example mapping 400 shown in FIG. 4 is an abbreviated for the sake of example and other mappings can include a significantly greater quantity of codes.

As described above, a system (e.g., data transformation system 101) can obtain transformation parameters (e.g., transformation parameters 105) via a computing device (e.g., computing device 103). The transformation parameters can specify the source data set (e.g., source data set 107), the source data schema 405, the destination data schema 409, and an intent of the target data set (e.g., target data set 113) can be to clinical patient data. Using the transformation parameters, the example system can obtain information of the source data schema 405, the target data schema 405, and the mapping 400. Based on the information received via the user interface, the system can transform the target data set from HL7 to FHIR.

An example code included in the source data set can be a negative test result from a clinical test encoded as: N\hat{ }Neg\hat{ }Cerner_observation_result\hat{ }2.16.840.1.113883.3.13. As shown in the source data schema 405 of mapping 400, “N” is a code identifier (CWE.1), “NEG” is result text (CWE.2), “observation result” is a coding system description (CWE.3), and 2.16.840.1.113883.3.13 is coding system identifier (CWE.6). Using the transformation process and the mapping 400, the system can transform the above HL7 code into corresponding a FHIR code: coding.code [system: 2.16.840.1.113883.3.13; code: N; display: NEG] of the target data schema 409.

Based on the FHIR codes generated by the transformation, the system determines a consumability score (e.g., consumability score 115) by determining transformation characteristics (e.g., accuracy, integrity, fidelity, usability, and validity). The system can assign an accuracy score of 0 if there is no structural mapping exists between a code in the source data set and the code of the target data set. The system can assign a score of 1 if there exists a structural mapping. As such, in the above example code, the system assigns a score of 1 because above HL7 code is structurally mapped to the FHIR code in the mapping 400.

Additionally, the system can determine an integrity score based on whether there is a structural mapping between elements of the code in the source schema and target schema. The system can assign an integrity score of 0 if there is no structural mapping and a score of 1 if there exists a structural mapping. In the above example, the source code includes three elements: code, system, and text, which structurally map to elements: code, system, and display in the target code. As such, in the present example, the system would assign an integrity score of 1 out of 1 (e.g., 1/1) because the above elements of the HL7 code are structurally mapped to components the FHIR code.

Furthermore, the system can determine a fidelity score based on whether the target data set uses a different encoding dictionary than the source data set. The system can assignee a fidelity score of 0 if information in the target data set uses a different encoding dictionary and a score of 1 if the dictionary is the same, In the above example code, the value 2.16.840.1.113883.3.13 is a code from a SNOMED used by both the HL7 data set and the FHIR dataset. Accordingly, in the present example, the system would assign an integrity score of 1/1.

Moreover, the system can determine a usability score based on whether the one or more components of the target data set have a different meaning than in the source data set. The system can assign a usability score if of 0 if the component in FHIR code has a different meaning than the component of the HL7 code and a score of 1 if the meaning is the same. In the above example, the HL7 data, NEG, is represented as text and the corresponding FHIR data, “display” is also text. Accordingly, in the present example, the system would assign an integrity score of 1/1.

Also, the scoring of usability can also be based on a binding strength of the transformed data in the destination schema. In the present example, the score can be a value between 0 and 1. In other cases, such as binding strength, the score assigned in a range (e.g., required=1, extensible=0.5, sample=0.1, and otherwise=−1). Assuming the values are determined to be extensible, the system would assign a value of 0.5/1.

The consumability score (CS) can be determined combining the scores to calculate an aggregated value. In the above example, assuming all the scores are weighted equally, the example consumability score of the target code would be (1+1+1+0.5)/4=87.5%. Based on the consumability score, the system determines recommendations for consuming the target data by comparing the consumability score determined to the thresholds (e.g., thresholds 219). In the present example, the thresholds define high quality data as having consumability score of 85% and above, fragmented data as having a consumability score of 70 to 85%, low quality data has a consumability score of 50 to 70%, and unusable data as having a consumability score of less than 50%. Recommendations for use target data set for the given intent can be provided based on the data quality. For example, if the intent is of the target data set is clinical decision making and the system can recommend the data for the given intent based on the high quality of the target data set (85% and above).

Different intents can have different acceptable thresholds. For example, if the target data set is used for billing, instead of clinical evaluation, then the system can recommend usage of the target data set having a consumability score greater than or equal to 70%.

Additionally, some recommendations can require combinations of characteristics of the target data set to meet a threshold. For example, the system can recommend the target data set for regulatory or public reporting purposes if the target data set has a consumability score greater than or equal to 50% and a round trip score of 1.

Further, the system can recommend improvements to the target data set based on the quality of the data. For example, the system can recommend automated reconciliation for high quality data (85% and above); manual reconciliation (data required human review, intervention) for fragmented data (70-85%); manual data translation, new data artifact creation (less than 50%). The consumability score improvement recommendation can also be based particular characteristics of the target data set. For example, if the fidelity score is below a threshold, the system can recommend standardizing reference vocabulary or using a standard terminology semantic equivalent (i.e., use NLP) match for the given proprietary coding system and code.

### 6. HARDWARE OVERVIEW

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

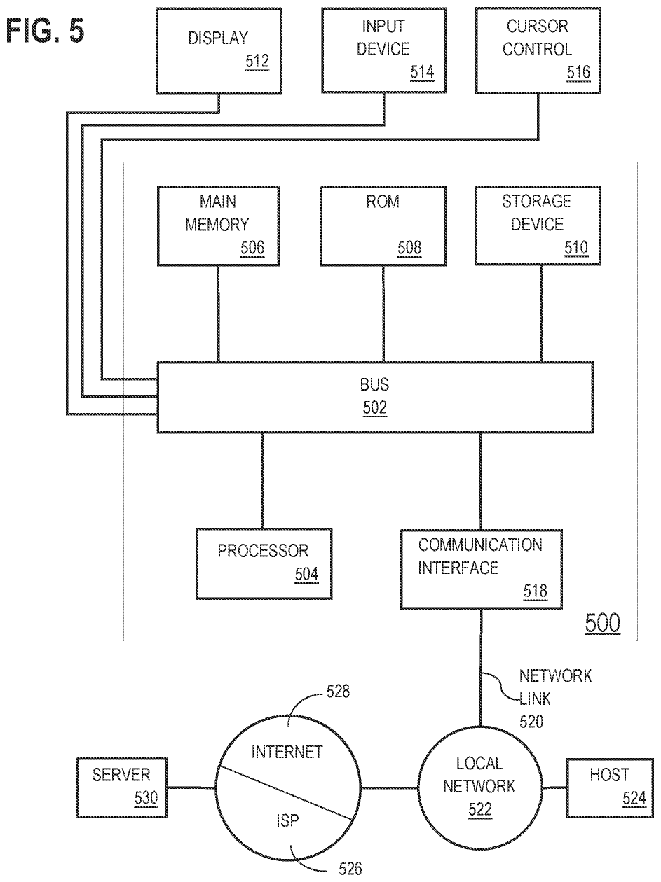

For example, FIG. 5 is a block diagram that illustrates a computer system 500 upon which an embodiment of the invention may be implemented. Computer system 500 includes a bus 502 or other communication mechanism for communicating information, and a hardware processor 504 coupled with bus 502 for processing information. Hardware processor 504 may be, for example, a general purpose microprocessor.

Computer system 500 also includes a main memory 506, such as a random access memory (RAM) or other dynamic storage device, coupled to bus 502 for storing information and instructions to be executed by processor 504. Main memory 506 also may be used for storing temporary variables or other intermediate information during execution of instructions to be executed by processor 504. Such instructions, when stored in non-transitory storage media accessible to processor 504, render computer system 500 into a special-purpose machine that is customized to perform the operations specified in the instructions.

Computer system 500 further includes a read only memory (ROM) 508 or other static storage device coupled to bus 502 for storing static information and instructions for processor 504. A storage device 510, such as a magnetic disk or optical disk, is provided and coupled to bus 502 for storing information and instructions.

Computer system 500 may be coupled via bus 502 to a display 512, such as a cathode ray tube (CRT), for displaying information to a computer user. An input device 514, including alphanumeric and other keys, is coupled to bus 502 for communicating information and command selections to processor 504. Another type of user input device is cursor control 516, such as a mouse, a trackball, or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

Computer system 500 may implement the techniques described herein using customized hard-wired logic, one or more ASICs or FPGAs, firmware and/or program logic which in combination with the computer system causes or programs computer system 500 to be a special-purpose machine. According to one embodiment, the techniques herein are performed by computer system 500 in response to processor 504 executing one or more sequences of one or more instructions contained in main memory 506. Such instructions may be read into main memory 506 from another storage medium, such as storage device 510. Execution of the sequences of instructions contained in main memory 506 causes processor 504 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 510. Volatile media includes dynamic memory, such as main memory 506. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 502. Transmission media can also take the form of acoustic or light waves, such as those generated during radio-wave and infra-red data communications.

Various forms of media may be involved in carrying one or more sequences of one or more instructions to processor 504 for execution. For example, the instructions may initially be carried on a magnetic disk or solid state drive of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector can receive the data carried in the infra-red signal and appropriate circuitry can place the data on bus 502. Bus 502 carries the data to main memory 506, from which processor 504 retrieves and executes the instructions. The instructions received by main memory 506 may optionally be stored on storage device 510 either before or after execution by processor 504.

Computer system 500 also includes a communication interface 518 coupled to bus 502. Communication interface 518 provides a two-way data communication coupling to a network link 520 that is connected to a local network 522. For example, communication interface 518 may be an integrated services digital network (ISDN) card, cable modem, satellite modem, or a modem to provide a data communication connection to a corresponding type of telephone line. As another example, communication interface 518 may be a local area network (LAN) card to provide a data communication connection to a compatible LAN. Wireless links may also be implemented. In any such implementation, communication interface 518 sends and receives electrical, electromagnetic or optical signals that carry digital data streams representing various types of information.

Network link 520 typically provides data communication through one or more networks to other data devices. For example, network link 520 may provide a connection through local network 522 to a host computer 524 or to data equipment operated by an Internet Service Provider (ISP) 526. ISP 526 in turn provides data communication services through the world wide packet data communication network now commonly referred to as the “Internet” 528. Local network 522 and Internet 528 both use electrical, electromagnetic or optical signals that carry digital data streams. The signals through the various networks and the signals on network link 520 and through communication interface 518, which carry the digital data to and from computer system 500, are example forms of transmission media.

Computer system 500 can send messages and receive data, including program code, through the network(s), network link 520 and communication interface 518. In the Internet example, a server 430 might transmit a requested code for an application program through Internet 528, ISP 526, local network 522 and communication interface 518. The received code may be executed by processor 504 as it is received, and/or stored in storage device 510, or other non-volatile storage for later execution.

### 7. MISCELLANEOUS; EXTENSIONS

Embodiments are directed to a system with one or more devices that include a hardware processor and that are configured to perform any of the operations described herein and/or recited in any of the claims below.

In an embodiment, a non-transitory computer readable storage medium comprises instructions which, when executed by one or more hardware processors, causes performance of any of the operations described herein and/or recited in any of the claims.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

While the forgoing description is directed to clinical data, it is understood that embodiments can be applied to other types of data encoded in compliance with predefined data schema. For example, embodiments can transform information and code encoded using predefined schemes

What is claimed is:

1. A non-transitory computer readable medium comprising instructions which, when executed by one or more hardware processors, causes performance of operations comprising:
identifying a transformation process for transforming data from a source clinical standard to a target clinical standard;
based on the transformation process and characteristics of a source data set corresponding to the source clinical standard, determining characteristics of a target data set corresponding to the target clinical standard that can be generated from the source data set; and
determine a consumability score for the target data set by evaluating the characteristics of the target data set based on a consumability criteria.

2. The non-transitory computer readable medium of claim 1, wherein the operations further comprise:
comparing the consumability score to a threshold; and
responsive to determining that the threshold is met, recommend consuming the target data set, consuming the target data set comprising using the target data set for a defined purpose of a user.

3. The non-transitory computer readable medium of claim 1, wherein the operations further comprise:
comparing the consumability score to a threshold; and
responsive to determining that the threshold is not met, recommend not consuming the target data set.

4. The non-transitory computer readable medium of claim 1, wherein the operations further comprise:
comparing the consumability score to a threshold; and
responsive to determining that the threshold is not met, recommend clinician review.

5. The non-transitory computer readable medium claim 1, wherein the operations further comprise:
comparing the consumability score to a threshold; and
responsive to determining that the threshold is not met, identifying factors a characteristic of the source data set that result in the greatest reduction of the consumability score.

6. The non-transitory computer readable medium of claim 1, wherein consumability criteria comprises one or more of: accuracy, integrity, fidelity, usability, and validity.

7. The non-transitory computer readable medium of claim 1, wherein the consumability score indicates a suitability of the target data set for use in making clinical decisions.

8. The non-transitory computer readable medium of claim 1, wherein the consumability score indicates the target data set affects efficacy of clinical decisions involving the target data set.

9. The non-transitory computer readable medium of claim 1, wherein the consumability score indicates a suitability of the target data set for a particular functionality.

10. A method comprising:
identifying a transformation process for transforming data from a source clinical standard to a target clinical standard;
based on the transformation process and characteristics of a source data set corresponding to the source clinical standard, determining characteristics of a target data set corresponding to the target clinical standard that can be generated from the source data set; and
determine a consumability score for the target data set by evaluating the characteristics of the target data set based on a consumability criteria.

11. The method of claim 10, further comprising:
comparing the consumability score to a threshold; and
responsive to determining that the threshold is met, recommend consuming the target data set, consuming the target data set comprising using the target data set for a defined purpose of a user.

12. The method of claim 10, further comprising:
comparing the consumability score to a threshold; and
responsive to determining that the threshold is not met, recommend not consuming the target data set.

13. The method of claim 10, further comprising:
comparing the consumability score to a threshold; and
responsive to determining that the threshold is not met, recommend clinician review.

14. The method of claim 10, further comprising:
comparing the consumability score to a threshold; and
responsive to determining that the threshold is not met, identifying factors a characteristic of the source data set that result in the greatest reduction of the consumability score.

15. The method of claim 10, wherein consumability criteria comprises one or more of: accuracy, integrity, fidelity, usability, and validity.

16. The method of claim 10, wherein the consumability score indicates a suitability of the target data set for use in making clinical decisions.

17. The method of claim 10, wherein the consumability score indicates the target data set affects efficacy of clinical decisions involving the target data set.

18. The method of claim 10, wherein the consumability score indicates a suitability of the target data set for a particular functionality.

19. A system comprising a hardware processor and computer-readable program instructions that, when executed by the hardware processor, control the system to perform operations, comprising:
identifying a transformation process for transforming data from a source clinical standard to a target clinical standard;
based on the transformation process and characteristics of a source data set corresponding to the source clinical standard, determining characteristics of a target data set corresponding to the target clinical standard that can be generated from the source data set; and
determine a consumability score for the target data set by evaluating the characteristics of the target data set based on a consumability criteria.

20. The system of claim 19, wherein the operations further comprise:
comparing the consumability score to a threshold; and
responsive to determining that the threshold is met, recommend consuming the target data set, consuming the target data set comprising using the target data set for a defined purpose of a user.

21. The system of claim 19, wherein the operations further comprise:
comparing the consumability score to a threshold; and
responsive to determining that the threshold is not met, recommend not consuming the target data set.

22. The system of claim 19, wherein the operations further comprise:
comparing the consumability score to a threshold; and
responsive to determining that the threshold is not met, recommend clinician review.

23. The system of claim 19, wherein the operations further comprise:
comparing the consumability score to a threshold; and
responsive to determining that the threshold is not met, identifying factors a characteristic of the source data set that result in the greatest reduction of the consumability score.

24. The system of claim 19, wherein consumability criteria comprises one or more of: accuracy, integrity, fidelity, usability, and validity.

25. The system of claim 19, wherein the consumability score indicates a suitability of the target data set for use in making clinical decisions.

26. The system of claim 19, wherein the consumability score indicates the target data set affects efficacy of clinical decisions involving the target data set.

27. The system of claim 19, wherein the consumability score indicates a suitability of the target data set for a particular functionality.

28. A non-transitory computer readable medium comprising instructions which, when executed by one or more hardware processors, causes performance of operations comprising:
obtaining a plurality of training data sets corresponding respectively to a plurality of transformation processes for transforming data between clinical standards, wherein a particular training data set in the plurality of training data sets comprises:
characteristics of a particular source data set corresponding to a source clinical standard;
characteristics of a target source data set corresponding to a target clinical standard that may be generated from the particular source code;
a consumability score for the transformation;
training a machine learning model using the training data sets to determine consumability scores;
apply the trained machine learning model to a current transformation to compute a first consumability score;
receive user input corresponding to the first consumability score; and
retraining the machine learning model based on the user input.

29. The non-transitory computer readable medium of claim 28, wherein the operations further comprise:
comparing the first consumability score to a threshold; and
responsive to determining that the threshold is met, recommend consuming the target data set.

30. The non-transitory computer readable medium of claim 28, wherein the operations further comprise:
comparing the first consumability score to a threshold; and
responsive to determining that the threshold is not met, recommend not consuming the target data set.
